(19) United States
(12) Patent Application Publication
(10) Pub. No.: US 2010/0049051 A1
(43) Pub. Date: Feb. 25, 2010

(54) Providing Hospital Information In A Portable Ultrasound System

(76) Inventors: Myung Hee Sang, Seoul (KR); Jin Yong Lee, Seoul (KR)

Correspondence Address: JONES DAY, 222 EAST 41ST ST, NEW YORK, NY, 10017, US

(21) Appl. No.: 12/612,168

(22) Filed: Nov. 4, 2009

(30) Foreign Application Priority Data
Apr. 11, 2008 (KR) 10-2008-0108717

(51) Int. Cl. A61B 8/14

(52) U.S. Cl. 600/443

(57) ABSTRACT

Embodiments for providing hospital information in a portable ultrasound system are disclosed. The portable ultrasound system includes a position information receiving unit to receive position information of the portable ultrasound system from an external system (e.g., GPS). A storage unit stores a table associating a plurality of diagnostic applications with respective diagnostic departments, and hospital information including hospital location information and diagnostic department information of a plurality of hospitals. A hospital information extracting unit searches the storage unit to find at least one hospital having a diagnostic department corresponding to an executed diagnostic application, and extracts hospital information corresponding to a hospital nearest to the portable ultrasound system among the at least one hospital based on the position information of the portable ultrasound system and the location information of the found hospitals. The portable ultrasound system displays location information on a screen of a display unit based on the extracted hospital information.

The present application claims priority from Korean Patent Application No. 10-2008-0108717 filed on Nov. 4, 2008, the entire subject matter of which is incorporated herein by reference.

### TECHNICAL FIELD

The present disclosure generally relates to ultrasound systems, and more particularly to a portable ultrasound system and a method of providing hospital information based on a diagnostic application executed therein and a location thereof.

### BACKGROUND

An ultrasound diagnostic system has become an important and popular diagnostic tool since it has a wide range of applications. Specifically, due to its non-invasive and non-destructive nature, the ultrasound diagnostic system has been extensively used in the medical profession. Modem high-performance ultrasound diagnostic systems and techniques are commonly used to produce two or three-dimensional diagnostic images of internal features of an object (e.g., human organs).

Recently, the ultrasound systems have been developed into two types of systems, namely, a cart-based ultrasound system and a portable ultrasound system similar to a laptop computer. The portable ultrasound system is often used for emergencies at an ambulance and the like, whereas the cart-based ultrasound system is typically used in hospitals. In an emergency situation, it is required to immediately transport a patient to a hospital for emergency treatment. However, it is not easy to find a hospital suitable for the patient in the emergency condition. Thus, there is a strong need for a portable ultrasound system, which can provide information on suitable hospitals closest to the patient.

### SUMMARY

Embodiments for providing hospital information in a portable ultrasound system are disclosed herein. In one embodiment, by way of non-limiting example, a portable ultrasound system comprises: an ultrasound diagnostic unit configured to execute one of a plurality of diagnostic applications to form an ultrasound image representative of a target object based on said executed diagnostic application; a position information receiving unit operable to receive position information of the portable ultrasound system from an external position information providing system; a storage unit to store a table associating the plurality of diagnostic applications with respective diagnostic departments, and hospital information including hospital location information and diagnostic department information of a plurality of hospitals; and a hospital information extracting unit operable to search the storage unit to identify at least one hospital having a diagnostic department corresponding to the executed diagnostic application, and extract hospital information corresponding to an identified hospital nearest to the portable ultrasound system based on the position information of the portable ultrasound system and the hospital location information of the identified at least one hospital.

In another embodiment, there is a method of providing hospital information in a portable ultrasound system that includes an ultrasound diagnostic unit configured to execute one of a plurality of diagnostic applications to form an ultrasound image representative of a target object according to the executed diagnostic application; a position information receiving unit operable to receive position information of the portable ultrasound system from an external position information providing system; a storage unit to store a table associating the plurality of diagnostic applications with respective diagnostic departments and hospital information including hospital location information and diagnostic department information of a plurality of hospitals; and a display unit to display the ultrasound image. Such a method comprises the following steps: searching the storage unit to identify at least one hospital having a diagnostic department corresponding to the executed diagnostic application; extracting hospital information corresponding to an identified hospital nearest to the portable ultrasound system based on the position information of the portable ultrasound system and the location information of the at least one identified hospital; and displaying location information based on the extracted hospital information.

The Summary is provided to introduce a selection of concepts in a simplified form that are further described below in the Detailed Description. This Summary is not intended to identify key or essential features of the claimed subject matter, nor is it intended to be used in determining the scope of the claimed subject matter.

### BRIEF DESCRIPTION OF THE DRAWINGS

FIG. 1 is a block diagram showing an illustrative embodiment of a portable ultrasound system.

FIG. 2 is a block diagram showing an illustrative embodiment of an ultrasound diagnostic unit.

FIG. 3 is a schematic diagram showing an example of a table in which diagnostic applications are associated with diagnostic departments.

### DETAILED DESCRIPTION

A detailed description may be provided with reference to the accompanying drawings. One of ordinary skill in the art may realize that the following description is illustrative only and is not in any way limiting. Other embodiments of the present invention may readily suggest themselves to such skilled persons having the benefit of this disclosure.

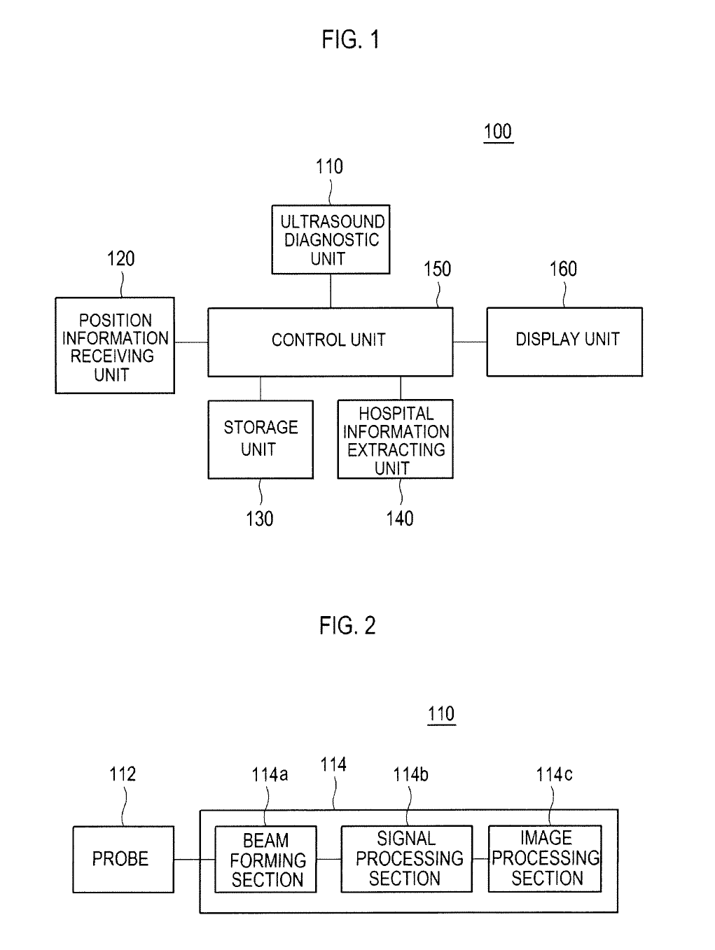

FIG. 1 is a block diagram showing an illustrative embodiment of a portable ultrasound system. Referring to FIG. 1, the ultrasound system 100 may include an ultrasound diagnostic unit 110 that may be operable to form ultrasound images representative of a target object. The ultrasound diagnostic unit 110 may execute one of a plurality of diagnostic applications that are installed on the portable ultrasound system 100. One of the diagnostic applications may be selected in response to a selection instruction inputted through an input unit (not shown). The diagnostic applications may include one or more of a fetal application for fetus diagnosis, a cardiac application for heart diagnosis, an abdominal application for abdomen diagnosis, a vascular application for blood vessel diagnosis, a muscular application for muscle diagnosis, a skeletal application for skeletal system diagnosis, an application for esophagus diagnosis, etc.

FIG. 2 is a block diagram showing an illustrative embodiment of the ultrasound diagnostic unit 110. As depicted therein, the ultrasound diagnostic unit 110 may include a probe 112 that may be operable to transmit ultrasound signals to the target object. The probe 112 may be further operable to receive echo signals reflected from the target object to thereby form electrical receive signals. The probe 112 may include an array transducer consisting of a plurality of elements. The array transducer may include a 1D or 2D array transducer, although it is not limited thereto. The array transducer may be operable to generate ultrasound signals and convert the echo signals into the electrical receive signals.

The ultrasound diagnostic unit 110 may further include a processor 114 coupled to the probe 112. The processor 114 may be operable to form an ultrasound image based on the electrical receive signals outputted from the probe 112. In one embodiment, the processor 114 may include a beam forming section 114a, a signal processing section 114b and an image processing section 114c.

The beam forming section 114a may be operable to apply delays to the electrical receive signals in consideration of positions of the transducer elements and focal points. The beam forming section 114a may be further operable to sum the delayed receive signals to thereby output a plurality of receive-focused beams. The signal processing section 114b may be operable to perform signal processing upon the receive-focused beams to thereby output ultrasound raw data. The processor 114 may further include an image processing section 114c. The image processing section 114c may be operable to form an ultrasound image based on the ultrasound raw data. The ultrasound system 100 may further include a position information receiving unit 120. The position information receiving unit 120 may be operable to receive position information on the portable ultrasound system 100 from external position information providing systems such as global positioning system (GPS) satellites. In one embodiment, the position information receiving unit 120 may include a GPS receiver to receive signals on position information from the GPS satellites to thereby compute a position of the portable ultrasound system 100 based on the receive signals.

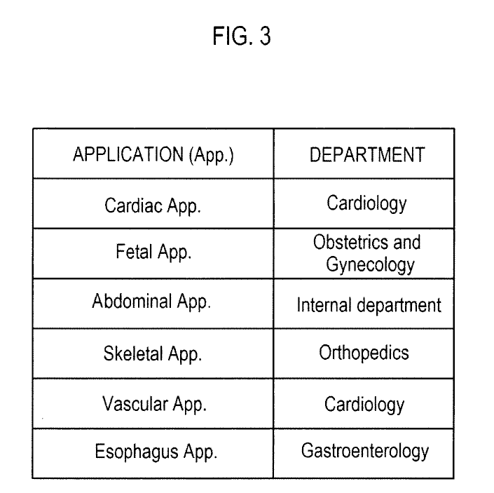

The ultrasound system 100 may further include a storage unit 130. In one embodiment, the storage unit 130 may store a table in which the diagnostic applications are associated with diagnostic departments, as shown in FIG. 3. Further, the storage unit 130 may further store hospital (or clinic) information. The hospital information may be stored in the storage unit 130 in association with the respective diagnostic departments. The hospital information may include location information, diagnostic department information, treatment time information, information on availability of an emergency room and the like.

The ultrasound system 100 may further include a hospital information extracting unit 140. The hospital information extracting unit 140 may be operable to extract information on a hospital nearest to the ultrasound system 100 from the storage unit 130 based on the selected diagnostic application and the position information of the portable ultrasound system 100. For example, the hospital information extracting unit 140 may be operable to identify hospitals in which a diagnostic department corresponding to the selected diagnostic application is available, from the storage unit 130. The hospital information extracting unit 140 may be further operable to select an identified hospital nearest to the ultrasound system 100 from the retrieved hospitals based on the position information of the ultrasound system 100 to thereby extract location information on the selected hospital.

The ultrasound system 100 may further include a control unit 150. The control unit 150 may be operable to control operations of the elements of system 100. For example, the control unit 150 may be operable to control the formation of the ultrasound image in the ultrasound diagnostic unit 110. Also, the control unit 150 may be operable to control a display unit 160 such that the ultrasound image and the extracted location information of the selected hospital are displayed on a screen of the display unit 160. The location information of the hospital may be displayed with text and/or a map, although the manner of displaying the location information is not limited thereto. Since the location information on the hospital nearest to the patient is displayed on the screen of the display unit 160, the patient may be quickly transported to the suitable hospital.

Although embodiments have been described with reference to a number of illustrative embodiments thereof, it should be understood that numerous other modifications and embodiments can be devised by those skilled in the art that will fall within the spirit and scope of the principles of this disclosure. More particularly, numerous variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. A portable ultrasound system, comprising:
an ultrasound diagnostic unit configured to execute one of a plurality of diagnostic applications to form an ultrasound image representative of a target object based on said executed diagnostic application;
a position information receiving unit operable to receive position information of the portable ultrasound system from an external position information providing system;
a storage unit for storing a table associating the plurality of diagnostic applications with respective diagnostic departments, and hospital information including hospital location information and diagnostic department information of a plurality of hospitals; and
a hospital information extracting unit operable to search the storage unit to identify at least one hospital having a diagnostic department corresponding to the executed diagnostic application, and extract hospital information corresponding to an identified hospital nearest to the portable ultrasound system based on the position information of the portable ultrasound system and the hospital location information of the identified at least one hospital.

2. The portable ultrasound system of claim 1, further comprising a display unit for displaying the ultrasound image and location information based on the extracted hospital information.

3. The portable ultrasound system of claim 1, wherein the position information receiving unit includes a global positioning system receiver.

4. The portable ultrasound system of claim 1, wherein the hospital information includes treatment time information and information on whether an emergency room is serviced.

5. The portable ultrasound system of claim 1, wherein the plurality of diagnostic applications includes one or more of the following: a fetal application for fetus diagnosis, a cardiac application for heart diagnosis, an abdominal application for abdomen diagnosis, a vascular application for blood vessel diagnosis, a muscular application for muscle diagnosis, a skeletal application for skeletal system diagnosis, and an application for esophagus diagnosis.

6. A method of providing hospital information in a portable ultrasound system including an ultrasound diagnostic unit configured to execute one of a plurality of diagnostic applications to form an ultrasound image representative of a target object based on the executed diagnostic application, a position information receiving unit operable to receive position information of the portable ultrasound system from an external position information providing system, a storage unit to store a table associating the plurality of diagnostic applications with respective diagnostic departments and hospital information including hospital location information and diagnostic department information of a plurality of hospitals, and a display unit to display the ultrasound image, the method comprising:
searching the storage unit to identify at least one hospital having a diagnostic department corresponding to the executed diagnostic application;
extracting hospital information corresponding to an identified hospital nearest to the portable ultrasound system based on the position information of the portable ultrasound system and the location information of the identified hospital; and
displaying location information based on the extracted hospital information.
